(19) United States
(12) Patent Application Publication
(10) Pub. No.: US 2021/0137520 A1
(43) Pub. Date: May 13, 2021

(54) ELASTOMERIC SUTURE

(71) Applicant: Asfora IP, LLC, Sioux Falls, SD (US)

(72) Inventors: Wilson Theophilo Asfora, Sioux Falls, SD (US); Michael Edward Villalta, Yerington, NV (US)

(21) Appl. No.: 16/947,541

(22) Filed: Aug. 5, 2020

Related U.S. Application Data
(60) Provisional application No. 62/960,309, filed on Jan. 13, 2020.
(60) Provisional application No. 62/882,835, filed on Aug. 5, 2019.

(51) Int. Cl. A61B 17/06; A61F 2/46; A61B 17/88

(52) U.S. Cl. CPC A61B 17/06166; A61B 17/06004; A61B 2017/06176; A61B 17/8869; A61B 2017/0618; A61F 2/4611

(57) ABSTRACT

The disclosure is related to a suture that is configured to connect two or more tissue portions. The suture includes an elongate body. The elongate body can include a capture section extending along a longitudinal axis with a radial dimension sized to extend through an opening through tissue, and a proximal and distal capture elements protruding radially at the ends of the capture section. The capture elements can have a capture element radial dimension that is sufficiently larger than the filament radial dimension to prevent the capture elements from sliding into the opening to thereby capture the tissue therebetween on the capture filament section for suturing the tissue.

### CROSS-REFERENCE TO RELATED APPLICATIONS

The present application claims the benefit of U.S. Provisional Patent Application No. 62/882,835, filed Aug. 5, 2019, and U.S. Provisional Patent Application No. 62/960,309, filed Jan. 13, 2020, both of which are incorporated herein by reference in their entirety.

### TECHNICAL FIELD

This disclosure is in the field of medical devices. More specifically, this disclosure is in the field of medical sutures.

### BACKGROUND

Various types of sutures and other devices are known for attaching body tissues together to facilitate the healing process. Sutures typically include a filament attached to a needle, which is typically tied or adhered to an end of the filament. The needle is grasped with a tool, such as forceps and is pushed through tissue, drawing the filament through the resultant hole in the tissue. The suture is then tied to hold it secure, and any excess is cut off.

The quality of the suturing is dependent on the practitioner's skill, and can be affected by inconsistent application of tension and inconsistent knot tying. For example, tissue portions that are to be held together to heal, should be held with the correct amount of holding pressure imparted by the suture. If there is too much pressure, blood and other bodily fluids might not reach the suture site, thereby slowing the healing process, or worse causing death of the tissue portions. Alternately, if the holding force is too light, the tissue portions might not be held in contact with one another and might not heal together, or might heal in improper or undesired ways, and have additional scar tissue. Additionally, injured tissues tend to be inflamed or swollen as a result of edema or other conditions. As tissues heal, the inflammation and swelling typically subside, and sutures that were once adequately tight, might become loose. Solutions are needed that allow medical professionals to quickly, accurately, and consistently apply sutures to tissue portions with the correct amount of holding force.

### SUMMARY

In one embodiment, a suture is disclosed including an elongate body made of an elastomeric material that has a surgical needle connected to the elongated body and configured for piercing animal tissue. The elongated body of the suture can comprise filament extending along a longitudinal axis, having a filament radial dimension. The elongated body of the suture can also have a protrusion protruding radially from the filament, made of an elastomeric material, selected such that axial tension causes the protrusion to narrow.

In another embodiment, a suture is disclosed that can have an elongate body that includes a filament extending along a longitudinal axis, having a filament radial dimension that is sized to extend through a tissue opening through animal tissue. The elongate body can also have a protrusion connected to the filament protruding radially. The protrusion can have a radial dimension that is sufficiently larger than the filament radial dimension. The protrusion can be made of an elastomeric material selected so that axial tension causes the distal capture element to narrow. The distal capture element can narrow to a narrowed radial dimension that is sufficiently reduced to allow the distal capture element to be pulled through the opening in the tissue. The distal capture element can return to the capture element radial dimension when the tension is released. The capture filament section can be positioned to move into the opening. In some embodiments, a piercing element can be connected to the elongate body and configured for piercing the tissue to make the opening.

In another embodiment, a suture is disclosed that can have an elongate body that includes a capture section that extends along a longitudinal axis. The capture section can have a filament radial dimension sized to extend through an opening through animal tissue. The elongate body can have proximal and distal capture elements protruding radially at the ends of the capture section. The capture elements can have a capture element radial dimension that is sufficiently larger than the filament radial dimension. The larger radial dimension of the capture elements can prevent the capture elements from sliding into the opening, so that the tissue is captured between the capture elements on the capture section for suturing the tissue. The distal capture element can be made of an elastomeric material selected so that axial tension causes the distal capture element to narrow. The distal capture element can narrow to a narrowed radial dimension that is sufficiently reduced to allow the distal capture element to be pulled through the opening in the tissue. The capture filament section can be positioned to move into the opening. In some embodiments, a piercing element can be connected to the elongate body and configured for piercing the tissue to make the opening. In some embodiments, the piercing element can be a surgical needle.

In other embodiments, the suture may have a distal capture element that includes a plurality of bent expansive portions that straighten elastically for contracting and bend elastically for re-expanding radially, and which are connect the distal filament section to the capture filament section. The distal capture element may also include a core that elastically maintains the plurality of expansive portions in a bent position.

In some embodiments, the elongate body includes a visual indicator that indicates when the distal capture element has passed through the opening and to indicate when the tissue is positioned between the capture elements.

In some embodiments, the elongate body can be made from an elastomeric material. In another embodiment, the proximal and distal capture elements of the suture can be capture nodules that have a capture nodule radial dimension that is sufficiently larger than the filament radial dimension to prevent the capture nodules from sliding into the opening in the tissue.

In another embodiment, a suture can have a manipulation feature extending along the longitudinal axis from the proximal capture nodule. In some embodiments, a manipulation nodule can protrude radially from the manipulation feature at a location proximal from the proximal capture nodule to provide purchase for a user to pull to apply the axial tension across the elongate body.

In another embodiment, the suture can have a holding structure located on the elongate body that defines a holding aperture. The holding aperture may have an inside dimension that sized to receive an opposite end of the elongate body therethrough to form the capture section. The protrusion may have a radial dimension such that the protrusion is capable of being pulled through the holding aperture, but sufficiently large to be retained by the holding structure against sliding back through the holding aperture to secure the suture.

In another embodiment, the suture may be part of a kit consisting of a suture and a cage, wherein the suture body is made of an elastomeric material and is configured to secure to and apply elastic tension to spinal processes of adjacent vertebrae and the cage configured to be implanted between and fuse together the adjacent vertebrae.

### BRIEF DESCRIPTION OF THE DRAWINGS

The drawings illustrate several embodiments, wherein identical reference numerals refer to identical or similar elements or features in different views or embodiments shown in the drawings.

FIG. 1 is a side view of an embodiment of a suture with a needle initially penetrating two tissue portions;

FIG. 2 is a side view of the suture of FIG. 1 illustrating tissue portions located in a tissue capture area, with the suture in a tensioned state;

FIG. 3 is a side view of the suture of FIG. 1 illustrating the tissue portions held within the tissue capture area, with the tension released;

FIG. 4 is a side view of another embodiment of a suture;

FIG. 5 is a side view of another embodiment of a suture, in the process of being inserted into two tissue portions, in a tensioned state;

FIG. 6 is a side view of the suture of FIG. 5, illustrating tissue portions located in a tissue capture area, with the tension released;

FIG. 7A is an isometric view of another embodiment of a suture with a fluted nodule;

FIG. 7B is a side view of the suture of FIG. 7A in a tensioned state;

FIG. 8 is an isometric view of another embodiment of a suture with a fluted nodule and a core;

FIG. 9 shows a molding insert suitable for forming the fluted nodule of FIGS. 7A and 7B;

FIGS. 10A and 10B are side views of another embodiment of a suture that can be placed in a looped configuration;

FIGS. 11A and 11B are side views of another embodiment of a suture that can be placed in a loop configuration;

FIGS. 12-16 are side views of a kit including the suture of FIGS. 11A and 11B and a cage, used in a procedure in the cervical spine; and

FIG. 17 shows a medical device attached to tissue using sutures as shown in FIGS. 11A and 11B.

### DETAILED DESCRIPTION

Embodiments of sutures described herein are configured and dimensioned holding tissue, such as during surgery and for restoration of tissue subjected to a traumatic or surgical injury. The sutures can be suitable for use in animal tissues, including the tissue of humans and other animals. Some embodiments are suitable for use with soft tissues, such as skin, muscle, tendon, ligament, dura, organs, etc., and some embodiments are configured to suture hard tissues such as bone. Some suture embodiments are suitable for use to suture or otherwise attach implants, medical devices, or prostheses to tissues. Some embodiments reduce or eliminate the need for using knots, thereby simplifying suturing procedures, while achieving more consistent results, with less influence from the skill of the practitioner.

Sutures described in this disclosure include an elongate body 101, which comprises a flexible filament 106 with a filament section 121 extending along the longitudinal axis 160, and may further comprise protrusions 108,110 with a radial dimension larger than a radial dimension of the filament 106 and/or additional features such as a holding structure 219 or manipulation feature 214. The filament section 121 can be a filament section adapted to capture an object, such as tissue or a medical implant or other device. The filament section 121 can be adapted to capture other items, such as features of medical devices like drug infusion pumps, screws, cages or other implants. In some embodiments, a filament section 121 is adapted to capture both tissue and medical devices such as implants at different locations along the longitudinal axis. The filament section 121 can be referred to as a “capture section.” In embodiments in which the filament section captures body tissue alone or in addition to artificial objects, it can be referred to herein as a “tissue capture filament section.” In some embodiments, the tissue capture filament section is flanked at either end by protrusions 108,110, which are adapted to capture both tissue and medical devices such as implants. In other embodiments, the tissue capture filament section is flanked at one end by a protrusion 108 and the other end by a different structure such as a holding structure 219 or manipulation feature 214, for example. The protrusions 108,110 can be referred to as a “capture elements.” In embodiments in which the protrusions 108,110 capture body tissue alone or in addition to artificial objects, they can be referred to herein as “tissue capture elements.” Capture elements and capture sections can define capture areas therebetween. In embodiments in which the protrusions capture areas capture body tissue alone or in addition to artificial objects, they can be referred to herein as “tissue capture areas.” The tissue capture area 112 can include the tissue capture filament section 121, and the tissue capture protrusion inner portions 109,111 that are facing the tissue capture filament section 121.

Referring to FIGS. 1-3, suture 100 has an elongate body 101, which includes a flexible filament 106 with a filament section 121 extending along the longitudinal axis 160, and protrusions 108,110 with a radial dimension larger than a radial dimension of the filament 106. The filament section 121 can be a filament section adapted to capture an object, such as tissue or a medical implant or other device. The filament section 121 can be adapted to capture other items, such as features of medical devices like drug infusion pumps, screws, cages or other implants. In some embodiments, a filament section 121 is adapted to capture both tissue and medical devices such as implants at different locations along the longitudinal axis. The body 101 has a portion that is configured to capture tissue, a device, and/or another structure, referred to as a “capture area”. In this embodiment, the capture area 112 includes a filament section 121 referred to as a “capture filament section,” which is flanked at both ends by protrusions 108,110, which act as capture elements as they are adapted to capture tissue and/or medical devices, such as implants, therebetween on the capture filament section 121. In embodiments in which the protrusions 108,110 capture body tissue alone or in addition to artificial objects, they can be referred to herein as “tissue capture elements.” Capture elements and capture sections can define capture areas therebetween. In embodiments in which the protrusions capture areas capture body tissue alone or in addition to artificial objects, they can be referred to herein as “tissue capture areas.” The tissue capture area 112 in FIG. 1 includes the tissue capture filament section 121, and the portions 109,111 of the tissue capture nodules 108,110 that are facing the tissue capture filament section 121, which cooperatively capture the patient tissue.

Protrusions 108,110 extend radially from the longitudinal axis 160 of the filament 106 with a sufficient radial dimension and stiffness that the protrusions will resist pulling through an opening in tissue. In the suture 100, the protrusions 108,110 are tissue capture elements 108,110 extending radially from the elongate filament 106. In other embodiments, other protrusions can be provided for other functions, such as manipulation protrusions to facilitate handling of the suture or holding protrusions to secure a portion of a suture to another portion of the suture.

In the embodiment of a suture 100, the tissue capture elements 108,110 are nodules that are proud of the filament 106 and positioned a distance 113 from one another along the flexible filament 106 between the distal end 102 and the proximal end 104 of the elongate body 101. The nodules 108,110 define a distal section 103, intermediate section 105, and a proximal section 107 of the elongate body 101. In this embodiment, the distal section 103 includes a distal filament section 119; the intermediate section 105 includes a tissue capture filament section 121; and the proximal section 107 includes proximal filament section 123. The proximal filament section 123 includes a manipulation feature 116.

The embodiment of suture 100 additionally includes a piercing element 124 attached at the distal end 102 of the distal portion 103 of the elongate body 101 and configured for piercing tissue portions 150,152 and drawing the elongate body 101 therethrough. Other embodiments are provided without a piercing element at the tip.

The elongated filament 106 has a filament radial dimension D1 perpendicular to the longitudinal axis 160, selected to enable the filament 106 to readily pass through an opening, hole, or incision 154 formed in a tissue portion 150,152 such as an incision 154 formed by the piercing element 124. The nodules 108,110 have a tissue capture radial dimension D2 greater than the radial dimension D1 of the elongated filament 106. The elongated filament 106 can be a monofilament, formed from a single strand. Alternatively, the elongated filament 106 can be formed from two or more filament components, for example in a woven or braided structure. The elongated filament 106 can be a composite of an elastomer and another material, or two or more different elastomers. The elongated filament 106 can be strands of different types of materials woven together. The elongated filament 106 can be a woven braid of strands of materials that are impregnated, overmolded, and/or encompassed by an elastomer. In some embodiments, the elongated filament 106 is formed with an inner core and an outer core of different materials.

As shown in FIGS. 1-3, the suture 100 has nodules 108,110 that are substantially spherical in shape when the suture 100 is not under tension T. The suture 100 has nodules 108,110 where the radial dimension of the nodules 108,110 varies along the axis 160 of the elongate filament 106. The slope and shape with which the nodules 108,110 rise away from the filament 106 can vary in different embodiments, and between different nodules 108,110. In the suture 100, the nodules 108,110 of the suture 100 rise abruptly away from the filament 106 in an arc-like fashion consistent with their spherical shape.

Other embodiments have a nodule that rises more gradually away from the filament 106, such as in a cone or ramp shape. Still other embodiments of sutures have a nodule that rises away from the filament 106 sharply, such as with nodules that are cubical or cylindrical. In other embodiments, a nodule has one side, (e.g., toward the distal end 102 of the suture) that rises away from the filament 106 with one slope, and another side (e.g., toward the proximal end 104 of the suture) with a different slope. For example, one embodiment of a suture has a nodule shaped like a cone, with the distal end of the nodule rising gradually away from the filament 106, and the proximal end of the nodule rising sharply from the filament 106. In such embodiments, the nodule has a surface facing the tissue capture filament section 121, which surface is sufficiently steep to a sufficient radial dimension to prevent the nodule to slide through the incision 154 when the tension is released. Other embodiments of sutures have nodules with other shapes that are adapted to pass through an incision 154 when the suture is under tension T and to capture tissue portion 150,152 when the tension T is released. In some embodiments, a nodule protrudes radially with an azimuth of 360 degrees, thereby protruding about the circumference of the suture. In other embodiments, a nodule protrudes radially with an azimuth of other angles, subtending lesser angles, such as a nodule that subtends an angle of 5 degrees, 90 degrees, 180 degrees, or 270 degrees, or the like. In other embodiments, a nodule is a U-shaped bend in the filament 106. In some embodiments, a nodule is L-shaped. In some embodiments, a nodule is solid. In some embodiments, a nodule is hollow. In some embodiments, a nodule is fluted, having an expansive portion that connects to longitudinal ends of adjacent sections of the elongate filament 106 that are separated by a longitudinal space. In other embodiments a fluted nodule has a core that connects longitudinal ends of adjacent sections. Other embodiments are envisioned that adapt sutures to various applications, types of tissues, implants or medical devices, or patients (e.g., pediatric, adult, animals, etc.).

In suture 100, nodules 108,110 are tissue capture nodules, configured to capture the sutured tissue portions 150,152 therebetween, and a capture area 112 is disposed therebetween, and consequently between the distal and proximal sections 103,107. The tissue capture area 112 can include a tissue capture filament section 121, and portions of the tissue capture nodules 108,110. The tissue capture area 112 has an untensioned length 113, one or more tensioned lengths 115 when being applied to a tissue portion 150,152, and one or more anchored lengths 117 when anchored in a tissue portion 150,152. In suture 100, the distal section 103 is defined between the nodule 108 and the distal end 102 of the elongate body 101. The proximal section 107 is defined between the nodule 110 to the proximal end 104 of the elongate body 101. In suture 100, the spacing of the nodules 108,110 along the axis 160 can be based on the type and/or thickness of the tissue portions 150,152 to be sutured. Additionally, in suture 100, the materials of the elongated filament 106 and/or the spacing of the nodules 108,110 can be configured based on the desired contact pressure or anchored dimension 117 between the tissue capture nodules 108,110. One or both of the nodules 108,110 protrude radially with a radial dimension D2 in a radially expanded position when the elongate body 101 is not under tension, and protrude a lesser amount in a radially contracted position when under tension with a narrowed radial dimension D3. Both of the dimensions D2 and D3 can be selected based on the type of tissue portions 150,152 to be sutured, for example by selecting the materials that form the nodules 108,110. In some embodiments, the tissue capture nodules can narrow under tension to a narrowed radial dimension D3 that is about 10% larger than the filament radial dimension D1 in an untensioned state.

The nodules 108,110 of suture 100 are both made of a material and configured so that they reduce in their protruding dimensions by about the same amount from D2 when radially expanded to D3 when radially contracted. In suture 100, the nodules 108,110 are formed from a material that deforms elastically in response to forces typically encountered in a suturing procedure. The material of the nodules 108,110 exhibits an elongation under tension, before rupture or yield. The properties, (e.g., the durometer, percent elongation, and Poisson ratio) of the nodules 108,110 material can be selected depending on the type or thickness of tissue portions 150,152 to be sutured. The nodules 108,110 can be configured to resist deformation when subjected to forces that are typical of forces encountered while a suture 100 is anchored in a tissue portion 150,152, such as forces caused by normal motion of the tissue portions 150,152 (e.g., a pulse, respiration, or swelling). The nodules 108,110 can be formed from similar materials that are suitable for forming the filament 106. In one embodiment, the nodules 108,110 are formed from silicone.

The nodules 108,110 can be fixed to the filament 106 by any suitable method. In one embodiment, the nodules 108,110 are overmolded on the filament 106 that extends continuously from the distal end 102 to the proximal end 104 of the elongate body 101. In another embodiment, the nodules 108,110 are adhered to the filament 106 such as with an adhesive, such as a glue. In another embodiment, the filament 106 and the nodules 108,110 are molded or cast as a single piece. In another embodiment, the filament 106 and the nodule 108 are co-extruded. The elongate body 101 can be formed from two or more co-axial elongated filaments 106 whose ends are joined together by the nodules 108,110.

Kits can be produced with a plurality of sutures 100 with different tissue capture area 112 lengths 113,117, i.e., different spacings between nodules 108,110 when untensioned (distance 113), and when anchored to tissue portions 150,152 (distance 117). The lengths 113,117 of the tissue capture area 112, or the range of lengths 113,117 provided in a kit for a particular type of tissue, can be selected based, for example, on whether the tissue is skin, dura, fascia, nerve, tendon, or ligament. For instance, a kit can be provided with embodiments of sutures 100 with a range of untensioned lengths 113 of the tissue capture area 112, from about 0.1 mm or 1 mm to about 5 mm or 1 cm. Other lengths can be used depending on the type of tissue and site intended for using the suture. An embodiment of a kit can contain embodiments of a suture 100 with untensioned lengths 113 of the tissue capture area 112 from about 0.2 mm to about 1.8 mm. Other kits can include other embodiments of sutures 100 with untensioned lengths 113 of the tissue capture area 112 that are above, or below this range, depending upon the type or thickness of tissue 150,152 to be sutured. For instance, a suture 100 adapted to repair an artery can have more closely spaced nodules 108,110 defining a tissue capture area 112, while a suture 100 adapted to repair skin on a knee can have more widely spaced nodules defining a tissue capture area 112.

In suture 100, the distal section 103 is configured to provide a location to grasp or manipulate the suture 100. The proximal section 107 also defines a manipulation feature 116 configured to provide purchase and a location to grasp, handle, or manipulate the suture 100 or to provide axial tension T. The manipulation feature 116 can be used to pull the suture 100 through the incision 154 to position the tissue capture filament section 121 to move into the incision 154. The proximal and distal sections 103,107 can be grasped by hands, fingers, forceps, or other tools to pierce the tissue portions 150,152, and to facilitate placement of the suture 100 therein. As described below, in other embodiments, other nodules define other sections and provide other functions.

The elongate body filament 106 of suture 100 can be entirely formed from a material that deforms elastically, or can have portions thereof that are made of such material. The material can be selected to significantly elongate and narrow under tension, before rupture or yield. In one embodiment, the elongated filament 106 can stretch elastically in response to an axial tension T by at least about 20%, 100% or 200% of its untensioned length, typically up to about 800%, 500%, or 300% of its untensioned length. In some embodiments, the tissue capture filament section 121 is selected with respect to the tissue 150,152 to be sutured so that the tissue capture filament section 121 remains stretched within the tissue 150,152 for example by between about 50% and 200% of its untensioned length 113.

The material and its configuration is selected so that the magnitude of forces under which the elongated filament 106 deforms is typical of what can be applied by a person, such as a surgeon or other medical professional, by hand, such as when gripping the suture 100 with their fingers or with forceps or other medical tools during a suturing procedure.

In some embodiments, the elongated filament 106 material can also be configured to deform elastically (stretch or shrink) when experiencing typical forces encountered when the suture 100 is anchored in a tissue portion 150,152 such as forces from motion of the tissue portion 150,152 (e.g., bending a knee or elbow with a suture in it). In some embodiments, the elongated filament 106 material can shrink or stretch elastically as forces due to swelling or edema decrease or increase respectively. For instance, the elongated filament 106 can shrink elastically to maintain a consistent contact pressure on the tissue portions 150,152 as a wound heals and tissue restores.

In some embodiments, an elastomeric suture is adapted to secure a craniotomy bone flap. If there is post-operative brain swelling; epidural, subdural, or intracerebral hematoma; hydrocephalus; or any other situation that causes the brain or associated tissues to expand, the elongated filament 106 can stretch elastically to allow expansion of the tissues, while allowing intracranial pressure to remain normal. This stretching can prevent brain injury to, or death of, the patient.

In some embodiments, an elastomeric suture is adapted to secure together two spinous processes following the fusion of adjacent vertebrae, for instance following a procedure such as posterior lumbar interbody fusion (“PLIF”) with a cage to promote lordosis. For instance, vertebrae are sometimes fused together to correct a number of spinal conditions. Such fusion can be accomplished using cages, rods, or other suitable devices. Typically those cages or rods are secured or connected with pedicle screws implanted in the vertebrae. In such embodiments, an elastomeric suture can keep compressive tension on the spinous processes to promote lordosis and prevent or lessen settling of the cage posteriorly. In other words, the elongated filaments 106 of the elastomeric suture can apply tension to adjacent spinous processes thereby imparting compression in the respective vertebrae bodies. Such use can be especially beneficial if a tapered drill is used to make space in the disc for a cage. Examples of spinal fusion systems and cages that can be used in the PLIF procedure are described, for example, in U.S. patent application Ser. No. 14/872,894.

In some embodiments, an elastomeric suture can be adapted to secure or tack a medical device, such as a subdermal or implantable drug infusion pumps, to internal tissue. Such pumps are frequently tacked to an abdominal wall with traditional sutures. These pumps frequently have a diameter on the order of 10 cm and a thickness on the order of 3 cm and are placed in a subcutaneous pocket created over the anterior abdominal wall. These pumps are often anchored to the underlying muscle fascia. When drug infusion pumps, for example pain medication delivery pumps, are tacked with traditional sutures, the sutures frequently break. Breakage can occur for a variety of reasons, including a patient colliding with an object, such as furniture or a wall, or even moving or straining in a certain way. When the sutures break, the pump can flip making it impossible to fill the pump because the pump refill port can be turned in toward the patient's body rather than facing outward adjacent the patient's skin. Additionally, drug delivery catheters that lead from such pumps to the drug delivery site can become kinked, twisted, or otherwise blocked preventing delivery of the drug. Such conditions can require additional surgery to correct. In contrast, if an implantable drug delivery pump is secured with an elastomeric suture, the elongated filaments 106 can elastically stretch and/or shrink without breaking and spring back to position after the movement or collision, thereby maintaining the desired position of the pump.

In some embodiments, an elastomeric suture can be adapted to close a patient's abdominal cavity following a laparotomy. If the patient experiences abdominal distention, for instance caused by ileus, elastomeric sutures can stretch as the abdomen distends, and then shrink as the condition subsides, keeping consistent pressure on the closed incision without causing the sutures to tear out.

In some embodiments, an elastomeric suture can be adapted for use as a skin expander. In cases where there is not enough skin to close a wound, surgeons traditionally use skin grafts. Using an elastomeric suture to pull on the opposing edges of a wound with a closing force, will cause the skin to grow and the wound edges to grow closer together. Over time, the wound will close. The tightness of the elastomeric sutures can be adjusted to maintain, increase, or decrease the closing force. Also, other elastomeric sutures of different lengths, elastic properties, and tissue capture regions can be placed in the wound edges over time to affect closing force.

The materials, properties, dimensions, and features of elastomeric sutures disclosed herein can be selected for a particular procedure or application. For example, in embodiments adapted for use to secure a craniotomy bone flap, the elastomeric sutures can have relatively narrow diameters D1, relatively long tissue capture area lengths 113, and/or can have a Young's modulus that allows them to stretch substantially in order to control intracranial pressure if a patient experiences brain swelling. In other embodiments, elastomeric sutures adapted for use in spinal fusion can have wider diameters D1, stiffer Young's moduli, and/or shorter tissue capture area lengths 113 allowing the elastomeric sutures to be adapted for the forces and tensions (e.g., compressive tension) required in spinal fusion. The adaptation of elastomeric sutures can be highly specific in some instances. For example, certain materials, properties, or dimensions of elastomeric sutures can be selected for fusing the L1 and L2 vertebrae, and others selected for fusing the L2 and L3 vertebrae. Additionally, the elastomeric sutures adapted for use in specific procedures can be further adapted for a specific indication. Returning to the example of elastomeric sutures adapted for use in securing a craniotomy bone flap, certain highly elastic sutures can be used if substantial brain swelling is expected, such as in the case of a patient who experienced traumatic brain injury, from an automobile crash. Different, less elastic sutures can be used if less swelling is expected.

The material forming the elongate body 101 or the more elastic portions thereof, can be made of a highly elastic material that exhibits a Poisson ratio, or the negative ratio of radial (or transverse) strain to axial strain such that when the material is stretched in an axial direction its radial dimension decreases. As shown in FIGS. 1 and 2, for the suture 100, when a tension T is applied along the axis 160 of the suture 100, the radial dimension D1 of the elongate filament 106 decreases to D4. The elongated filament 106 can be formed from polymers. In various embodiments, the polymers can be thermosets, thermoplastics, or elastomers. In various embodiments, the elongated filament 106 can be formed from elastomers such as natural or synthetic rubbers. In one embodiment, the elastomer forming the elongated filament 106 is a polysiloxane, such as silicone. In some embodiments, the radial dimension of the elongate body 101 or selected parts thereof decreases in the tensioned state at least down to about 15%, 20%, 30%, 50% or 60% of its untensioned radial dimension.

In one embodiment, the elongated filament 106, or typically portions of the elongate body 101 outside of the tissue capture area 112, is formed from a material that is relatively less elastic than the material of the nodules 108,110 or a stiff, inelastic material. In an embodiment, the tissue capture filament section 121 is more elastic than the proximal and/or distal filament sections 123,119 and the tissue capture filament section 121 can be as elastic as the distal and/or proximal tissue capture nodules 108,110. For example, the material of the filament 106 or selected portions thereof can strain less in an axial direction in response to an applied tension T than the material of the nodules 108,110 under the same tension T. In another embodiment, the filament 106 material exhibits less of a Poisson effect than the nodule 108,110 material. In another embodiment, a tissue capture nodule 108 has a higher Poisson ratio than another portion of the elongate body 101. In various embodiments, the filament 106 material, or sections thereof, is formed of the same class of materials as the nodules 108,110, and in one embodiment, the tissue capture area 112, including the tissue capture filament section 121 and tissue capture nodules 108,110 are made of a highly elastic material, different than the remainder of the elongate body 101. In various other embodiments, the filament 106 and the nodules 108,110 are formed from different materials.

In one embodiment, the elongate body 101 is formed from an absorbable material that will dissolve or incorporate over time. In various embodiments, the elastomeric material can be gut, polydioxanone, poliglecaprone, or polyglactin. In other embodiments, the filament 106 material is non-absorbable. In various embodiments, the filament 106 material can be nylon, polypropylene, silk, polyester, or silicone. In one embodiment, the nodules 108,110 are formed from silicone and the filament 106 formed from nylon. In another embodiment, the nodules 108,110 are formed from silicone having one durometer or hardness, and the filament 106 can be formed from silicone having a second durometer. In other embodiments, the durometer of the nodules 108,110 is less than that of the filament 106. In various embodiments, the nodules 108,110 are formed of a material such that the percent elongation and Poisson effect of the nodules 108,110 is greater than or less than that of the filament 106 under the same tension, T. In another embodiment, the nodules 108,110 can be formed from a material with a durometer greater than that of the filament 106.

Some suture embodiments have a visual indicator provided on one or both tissue capture nodules 108,110 or on other nodules, selected to be visually distinct from one another, or from tissue portions 150,152. Visual indicators can additionally or alternatively be provided on portions of the filament 106. In various embodiments the visual indicators are provided on the distal section 103, intermediate section 105, and/or proximal section 107. In various other embodiments, visual indicators can be applied to the distal filament section 119, the tissue capture filament section 121, and/or the proximal filament section 123. These visual indicators provide a visual indication of the tissue capture area 112 or its boundaries. In various embodiments, the visual indicators can include, for example, colors, patterns, or textures that enable a user of the suture 100 to differentiate the regions of the suture 100, such as a tissue capture area 112, or a manipulation feature 116. A visual indicator can enable a user to differentiate the nodules 108,110 and the elongated filament 106 from each other, or from the tissue portions 150,152 being sutured. A medical professional suturing the tissue portions 150,152 can tell when the distal tissue capture nodule 108 has passed through the tissue portions 150,152 and the proximal tissue capture nodule 110 has not, for instance, indicating that the tissue capture area 112 is properly positioned within the tissue portions 150,152 so that the tension and stretching of the elongated nodule can be released. Visual indicators on a manipulation feature 116 can also assist a medical professional to grasp the suture. The visual indicators can be adapted based on the type of tissue 150,152 being sutured. For instance, a tissue capture area 112 of a suture 100 adapted for use with dura (which is lightly colored) can be a dark or contrasting color.

A piercing element 124 is attached to the distal end 102 of the elongate body 101 of the suture 100. Referring to FIGS. 1-3 in the embodiment of suture 100, the piercing element 124 is elongated and has a sharp point 126 to pierce the tissue 150,152 and an attachment end 130 connected with the elongate body 101, and preferably with the filament 106. The piercing element 124 of suture 100 is a surgical needle. The embodiment of the suture 100 has a straight needle 124 with a round, substantially cylindrical cross-section. In some embodiments, the piercing element 124 has other cross-sections such as triangular, spatula (substantially flat), or rectangular, as desired, for the type or thickness of tissue to be pierced. In other embodiments, the cross-section of the piercing element 124 can be uniform along its length, or it can be non-uniform. In other embodiments, the piercing element 124 can be straight, it can be curved, or it can have some sections that are straight and others that are curved. In some embodiments, the piercing element 124 can have a cutting feature on a concave surface of a curve of the piercing element 124, or a reverse cutting feature on a convex portion of the piercing element 124. The point 126 can be adapted for use in certain tissues. In some embodiments, the point 126 is rounded, for instance for use in visceral or abdominal tissues. In other embodiments, the point 126 is triangular with penetrating characteristics, for instance for use with skin. In other embodiments, the point 126 is a diamond shape, with four sharp sides, for instance adapted for use in calcified or sclerous tissues. In other embodiments, the point 126 is blunt, for instance for use in parenchymal tissue. In various embodiments, the piercing element 124 is a needle sized in a range of gauges from about a 1/0 gauge, to about a 10/0 gauge. In other embodiments, the needle 124 can be larger or smaller than these typical sizes. The needle 124 can be made integral with the elongate body 101, such as by overmolding the filament 106 on the needle 124 or other methods known in the art, or can be attached in other manners, including releasably, such as by threading the elongate body 101 through the needle 124.

As shown in FIGS. 1-3, in use, the point 126 of the piercing element 124 is forced through tissue portions 150,152, thereby leaving an incision 154. As the suture 100 is passed through the tissue portions 150,152, a tension T is applied along the axis 160 of the suture 100. Tension T can be applied for instance by grasping the piercing element 124 with forceps and pulling the suture 100. Alternately, the suture 100 can be grasped by both the piercing element 124, the distal section 103, and/or the manipulation feature 116. The suture 100 can be grasped and tensioned by two forceps, or it can be grasped and tensioned by a specialized spreader tool. As illustrated in FIG. 2, the application of the tension T causes the radial dimension D2 of the nodules 108,110 to decrease relative to their untensioned radial dimensions D2 in a radially expanded position to a tensioned dimension D3 in a radially contracted position. The materials, shapes, and/or sizes of the nodules 108,110 are selected such that when the tension T that corresponds to tension typically encountered by a suture in a suturing procedure is applied to the suture 100, the dimension D3 is of a scale to allow the suture 100 to pass through the incision 154 without stretching, tearing, or otherwise damaging the tissue portions 150,152. The dimension D3 can be selected in concert with a radial dimension of the piercing element 124, such that the incision 154 created by the piercing element 124 is of an appropriate size to allow the nodules 108,110 with dimension D3 to pass through.

When the tissue portions 150,152 are located within the tissue capture area 112, the tension T can be released. As illustrated in FIG. 3, when the tension T is released, the radial dimension of one or both of the nodules 108 and 110 return to their untensioned values D2 in a radially expanded position and cooperate to capture and hold the tissue portions 150,152 in the tissue capture area 112. The distance between the nodules 108,110 decreases from distance 115 when the suture 100 was being applied, to the distance 117 where the nodules 108,110 apply pressure to the tissue portions 150,152. Excess material can be trimmed from the suture 100 after the tension T is released. For instance, the elongated filament 106 can be trimmed outside of the tissue capture area 112 including the end of the elongated filament 106 that holds the piercing element 124, and the portion of the elongated filament 106 defining the manipulation feature 116. The excess can then be discarded.

FIGS. 4-11B show other suture embodiments 200, 300, 400, 500, 700, and 800. Some materials, structure, and methods of using the sutures 200, 300, 400, 500, 700, and 800 can be substantially the same as the suture 100, further description of which would be cumulative and will be omitted for the sake of brevity.

Referring to FIG. 4, suture 200 has a manipulation feature 116 defined in the proximal section 107 of the elongate body 101. In suture 200, the manipulation feature 116 is defined between the tissue capture nodule 110 and a manipulation protrusion 214 located at the proximal end 104 of the elongate body 101. In the suture 200, the manipulation protrusion 214 is a manipulation nodule 214. The manipulation nodule 214 provides an additional feature for a medical professional to grasp the suture 200 with, thereby facilitating application of the suture 200. The manipulation nodule 214 can also provide a feature to facilitate removal of the suture 200, such as when a wound is sufficiently healed to no longer need the suture 200. For example, a surgeon or other medical provider can grasp the manipulation nodule 214 with forceps, finger, or other tools to remove it from the tissue portions 150,152. The manipulation nodule 214 can also provide a reference for a medical professional using the suture 200 to indicate which end of the suture 200 contains the penetration feature 124. In some suture 200 embodiments, the manipulation nodule 214 can be made from the same materials as the tissue capture nodules 108,110. In other suture 200 embodiments, the manipulation nodule 214 can be made of different materials than the tissue capture nodules 108,110.

FIGS. 5 and 6 illustrate another embodiment of a suture 300. While tissue capture nodules 108,310 of the suture 300 can be formed from a material that deforms elastically in response to forces typically encountered in a suturing procedure, others of the nodules 108,310 can be formed from materials that are rigid and do not appreciably deform under the forces typically encountered in a suturing procedure. In various embodiments, the nodules 108,310 can have different material properties, such as durometer, such that one nodule 108,310 stretches more than the other nodule 108,310 under the same tension T. For example, the nodule 108 can be made of a durometer that is softer than nodule 310, causing the nodule 108 to elongate and narrow more than nodule 310 under the same tension T.

As shown in FIG. 5, with the application of tension T, the nodule 108 narrows to a dimension D3 in a radially contracted position allowing the nodule 108 to pass through the incision 154. However, the nodule 310 has a radial dimension D2 that does not narrow with the application of tension T, and cannot pass through the incision 154. Such a suture 300 can be adapted to facilitate one handed insertion, as tension T can be applied for instance by grasping the piercing element 124, or the distal section 103 of the elongated filament 106, with the nodule 310 providing resistance against the tissue portion 152, thereby generating tension T in cooperation with the grasping of the suture 300, such as by forceps.

FIGS. 7A and 7B illustrate an embodiment of a suture 400 that has a protrusion that is a fluted nodule 408 formed from a plurality of expansive portions 402. The fluted nodule 408 protrudes radially with a radial dimension D2 in a radially extended position, such as when the elongate body 101 is not under tension. In the radially extended position, the expansive portions 402 are splayed out and separated from one another circumferentially by circumferential spaces 403. In this embodiment, the circumferential spaces 403 are flutes. The fluted nodule 408 protrudes a lesser amount in a radially contracted position when under tension, with a narrowed radial dimension D3. The longitudinal ends of expansive portions 402 connect to longitudinal ends of adjacent sections 119,121 of the elongate filament 106 that are separated by a longitudinal space 405. The expansive portions 402 elastically join the sections 119,121. The expansive portions 402 include arms 404 that protrude radially from the elongate filament 106. The arms 404 protrude from the elongate filament 106 radially at an angle with respect to the longitudinal axis 160 of the filament 106. In some embodiments a fluted nodule is a tissue capture element.

An arm 404 is joined to another arm 404 by an elbow 406. When the fluted nodule 408 is in a radially extended position, the elbows 406 are partially closed. When the fluted nodule 408 is in a radially contracted position, the elbows 406 are open, as shown in FIG. 7B. In the radially contracted position, the expansive portions 402 can contact one another and the flutes 403 can close.

The arms 404 can be integrally formed with the elongate filament 106, such as by co-molding or by otherwise attaching an arm 404 to the elongate filament 106. The arms 404 can be made from the same material as the elongate filament 106, or they can be made from other materials than the elongate filament 106. Likewise, the elbow 406 can be formed from the same material as the elongate filament 106 and/or the arms 404, and can be unitarily formed therewith. In this embodiment, the radial dimension D2 in the radially expanded position is measured at the outer surface of the expansive portions 402, at the elbow 406. The arms 404 and/or the elbows 406 cause the expansive portions 402 to deform when tension is applied longitudinally to the elongate filament 106, such that the fluted nodule 408 radial dimension D2 decreases to a radially contracted position with a dimension D3 when under tension. When the fluted nodule 408 is in the radially expanded position, the elbows are in a partially closed position, as shown for example, in FIG. 7A. As tension is applied to the elongate filament 106, the elbows 406 straighten, from the partially closed position, to an open position as shown for example in FIG. 7B. The opening of the elbows 406 under tension reduces the tissue capture element radial dimension D2 to a narrowed radial dimension D3 in the radially contracted position. In this embodiment, the elbows can also stretch, elongating under tension. In some embodiments, the arms 404 can stretch under tension.

The deformation of the arms 404 and/or elbows 406 is elastic, such that when a longitudinal tension is released from the elongate filament 106, the portions 404,406 return to their untensioned shapes, and the fluted nodule 408 returns to its radially expanded position.

In some embodiments, the expansive portions 402 are an elastic articulation having a continuous arc extending radially away from the elongate filament 106 at one end of the longitudinal space 405 and back to the elongate filament 106 at the other end of the longitudinal space 405. In some embodiments, the expansive portions 402 are elastic articulations having a concave curved section where they radially extend away from the elongate filament 106 at either end of the longitudinal space 405. The convex curves are joined by a concave curved section. The elastic articulation can stretch longitudinally in response to a longitudinal tension, and can straighten, causing the fluted nodule 408 to assume a radially contracted position

FIG. 8 illustrates an embodiment of a suture 500. The suture 500 is similar to the suture 400 but the fluted nodule 408, in addition to the expansive portions 402, includes a core 410 that spans the longitudinal space 405 between adjacent sections of the elongate filament 106, such as sections 119,121. In this embodiment, the core 410 is elastomeric. In some embodiments, the core 410 is unitary with the elongate filament 106, such as when the suture 400 is molded as a single piece. In other embodiments, the core 410 is an inner core of the elongate filament 106, extending along at least a portion of the length thereof. In some embodiments, the elongate filament 106 is formed with an inner core 410 and an outer layer of the elongate filament 106 is overmolded on the inner core 410. In some embodiments, the inner core 410 and the outer layer of the elongate filament 106 are formed with different materials. In various embodiments, the different materials have different mechanical properties, such as durometer, percent elongation, Poisson ratio, elastic modulus, or the like. For example, in some embodiments, the material of the inner core 410 is an elastomer with an elastic modulus that is stiffer than the elastic modulus of the outer layer. In other embodiments, the inner core 410 is an elastomer with an elastic modulus that is less stiff than the elastic modulus of the outer layer. In another embodiment, the core 410 is elastomeric, while the filament 106 is a non-elastomeric material. The core 410 can have a circular cross section, as shown in FIG. 8, although other cross-sections, such as squares, rectangles, or other polygons, ellipses, or irregular shapes are contemplated.

The core 410 maintains the fluted nodule 408 in a radially expanded position by contracting the longitudinal space 405, closing the elbows 406 and pushing the expansive portions 402 radially away from the elongate filament 106. Thus, the core 410 prevents the tissue capture portion 408 from pulling through the incision 154. For example, the core 410 retains the expansive portions 402 in the radially expanded position when tension is applied along the elongate filament 106 and that tension is resisted by contact of the expansive portions 402 against a tissue portion 150,152, such as when tissue portions swell, or a patient moves. A suture including a core 410 deforms less under an applied tension than an otherwise similar suture but without a core 410. In embodiments in which the core 410 is stiffer than the cumulative stiffness of the expansive portions 402, the core 410 resists deformation of the fluted nodule 408 when a longitudinal tension is applied to the elongate filament 106 and principally governs the elastic response of the fluted nodule 408 to the applied tension.

In the embodiments of FIGS. 7A, 7B, and 8, the fluted nodule includes six expansive portions 402. Other embodiments can have more or fewer expansive portions 402, preferably at least two. However, some embodiments have one expansive portion 402.

The mold insert 600 shown in FIG. 9 is suitable to mold a fluted nodule 408 of the embodiment shown in FIGS. 7A and 7B. The mold insert has a disc-shaped body 602 with a first face 608 separated from a second face 610 by the thickness of the body 602. The body 602 has a plurality of channels 604 extending into the first face 608 and second face 610. The channels 604 that extend into the first face 608 intersect at a confluence 612. Likewise the channels 604 that extend into the second face 610 intersect at a confluence 614. Each channel of the plurality of channels is separated from an adjacent channel by a land 606. The lands 606 are wedge-shaped. During molding of a suture with a fluted nodule, such as suture 400, the insert is placed inside a mold that defines the outer structure of the suture. The insert 600 has the shape of a negative image of the fluted nodule 408. As a material, such as an elastomer is injected into the mold, the material flows around the insert 600, filling the channels. After the material has cured or is sufficiently stable, the outer mold and the insert are removed, leaving the suture with the fluted nodule 408. In other embodiments, the insert has a central aperture connecting the confluences 612,614. Such an insert would be acceptable to mold a fluted nodule with a core, such as the nodule of FIG. 8.

In the embodiment of FIGS. 10A and 10B, suture 700 has a holding structure 219 located at the proximate end 104 of the suture, which in this embodiment defines a holding aperture 216. The holding aperture 216 is sized to receive the distal end 102 of the elastomeric suture 700 inserted therethrough to form a tissue capture filament section 121 such as a holding loop 218. In some embodiments, the distal end 102 has a piercing element 124, such as a needle. The distal end 102 can be used as a lasso and wrapped around, and can be passed through an incision or opening in tissue, a surgical tool, or implant, and looped back through the aperture 216 to secure such items within the holding loop 218.

The suture 700 in this embodiment has a plurality of elastomeric holding protrusions 702 to allow a user to progressively ratchet the protrusions 702 through and hold the holding aperture 216 thereby tightening the elongate filament 106 and securing the tissue, tool, or implant within the holding loop 218. In this embodiment, the holding protrusions 702 have a radial dimension larger than the inside of the holding aperture 216. The holding protrusions 702 of this embodiment are conical shapes that enables them to be easily inserted into the holding aperture 216, but resist being pulled through the aperture 216 in the opposite direction from insertion. For example, the base of the cone of the holding protrusion 702 is larger than the inside diameter of the holding aperture 216. In other embodiments the holding protrusions 702 are nodules such as spherical nodules, cubes, arrows, wedges, prisms, fluted nodules, or the like. Alternative embodiments have a tissue capture element extending radially from the elongate filament 106 to capture tissue portions.

In some embodiments, the holding aperture 216 is formed in a protrusion, such as a manipulation protrusion 214, or other nodule located at the proximate end 104 of the suture. In other embodiments, the holding structure 219 is a loop of the elongate filament 106, that forms the holding aperture 216 by passing a portion of the elongate filament 106 back on itself and securing the elongate filament 106 to itself. In other embodiments, the holding structure 219 is unitarily formed with the suture 700 to form the holding aperture 216 with the elongate filament 106 such as by using a suitable mold.

In some embodiments, the holding structure 219 is located at an end of the suture, such as suture 700, for example. In other embodiments, a holding structure 219 and holding aperture 216 are located at other positions along the length of the elongate filament 106. In some embodiments, a plurality of holding structures 219 are disposed along the length of the elongate filament 106.

In this embodiment, the holding structure 219 of the suture 700 forming the holding aperture 216 is made from an elastomeric material. As a protrusion 702 passes through the holding aperture 216, the conical shape of the holding protrusion 702 pushes the holding structure 219 radially outward, stretching it, thereby enlarging the aperture 216 and allowing the holding protrusion 702 to pass through. As the wide base of the holding protrusion 702 passes through the aperture 216, the holding structure 219 elastically returns to its previous size and shape. If a holding protrusion 702 that has passed through the aperture 216 is pulled back toward the aperture 216, the wide base of the holding protrusion 702 pushes on the holding structure 219 longitudinally and does not stretch the holding structure 219 radially, preventing the holding protrusion 702 from passing back through the aperture 216.

Both the holding protrusions 702 and the holding structure 219 are elastomeric in one embodiment. In other embodiments, the holding structure 219 is not elastomeric, and in some embodiments is not elastic. In other embodiments, the holding structure 219 is elastomeric but is stiffer than the holding protrusions 702. In such embodiments, as tension is applied along the elongate filament 106, the holding protrusions 702 stretch longitudinally and shrink radially to a contracted radial position with a dimension smaller than an internal dimension of the holding aperture 216, thereby allowing the holding protrusions 702 to pass through the holding aperture 216. When tension is released, the holding protrusions 702 return to their extended radial positions and prevent passage back through the holding aperture 216.

The holding structure 219 is typically adapted to elastically grasp a holding protrusions 702. For instance, a holding protrusions 702 can have a receiving feature, such as a slot or ring complementary to the shape and size of the aperture 216 formed in the holding structure 219 such that the holding structure 219 snaps into the receiving feature thereby securing the suture 700. Various embodiments of a suture 700 can have more or fewer holding protrusions 702 than shown in FIGS. 10A and 10B, and can have as few as one or zero holding protrusions 702.

In the embodiment of FIGS. 11A and 11B, suture 800 has a holding structure 219 located at the proximate end 104 of the suture, which in this embodiment defines a holding aperture 216. The holding aperture 216 is sized to receive the distal end 102 of the elastomeric suture 800 inserted therethrough to form a tissue capture filament section 121 such as a holding loop 218. The construction of suture 800 is similar to the construction of suture 700, shown in FIG. 10A, with rounded holding protrusions 812, and also, at its distal end 102, with a piercing element 124, such as a needle, although other embodiments end at a portion of the filament or have other constructions free of a needle. In one embodiment, the holding aperture 216 is inelastic and the protrusions 812 are elastic. In another embodiment, the holding aperture 216 is elastic and the protrusions 812 are elastic.

Some suture embodiments are configured for use in a spinal fusion, and some are provided as a component of a spinal process tensioner kit. Such a kit can be utilized, for example, to create a dynamic lordosis produced gradually after surgery. An example of such a kit can include a suture 800 and a cage 802, which cage 802 is adapted to be implanted in the disk space between adjacent vertebrae, as illustrated in FIGS. 12A-E. The distal end 102 of suture 800 can be configured as a lasso and wrapped around, and/or passed through openings in the spinal processes of adjacent vertebrae, and looped back through the aperture 216 to secure the spinal processes within the holding loop 218.

The suture 800 in this embodiment has a plurality of elastomeric holding protrusions 812 to allow a user to progressively ratchet the protrusions 812 through and hold the holding aperture 216 thereby tightening the elongate filament 106 and securing the spinal processes 830 within the holding loop 218, allowing the surgeon to select the desired elastic tension between the spinal processes. In this embodiment, the holding protrusions 812 have a radial dimension larger than the inside of the holding aperture 216. The holding protrusions 812 of this embodiment can be any shape that enables them to be easily inserted into the holding aperture 216, but resist being pulled through the aperture 216 in the opposite direction from insertion.

In some embodiments, a spinal fusion system may include a fusion cage 802. The cage shown in FIG. 15 has a substantially cylindrical shape defining a proximal end 814 and a distal tip 816, but other suitable shapes and configurations of cages and implants can be used, such as tapered cages or expandable cages. As illustrated in FIG. 15, the distal tip 816 can have a frustoconical shape. In this manner, the fusion cage may be generally bullet shaped 802. Additionally, the cage shown is free of fixation screws that affix the cage to the bone, thereby allowing easier pivoting of the vertebral bodies 844 and the cage in response to the tension applied by the elastomeric suture 800 bringing the spinal processes 830 together.

Referring to FIGS. 12-16, the suture 800 of FIG. 11 can be utilized with a bullet cage 802 as a kit to perform the PLIF procedure. The vertebrae may be spread using a contralateral tapered distractor 820 (FIG. 13) and the disk space 832 drilled 848 using a tapered drill 822 from the posterior of the cervical spine to expose cancellous bone tissue 824 at the posterior side of the vertebrae and leave the vertebrae cortex intact 826 at the anterior side (FIG. 15). The bullet cage 802 is then inserted posteriorly, either by threading or pushing, where it will easily penetrate and fix to the exposed cancellous bone 824 of the vertebrae and the suture 800 is installed on contralateral spinal processes 830 to the bullet cage 802 (FIG. 15), either by threading the suture 800 through an opening 836 in one of the spinal processes 830 and then looping the suture around the adjacent spinal process 830 as illustrated in FIGS. 15 and 16, or by treading the suture 800 through openings 836 in two adjacent spinal processes 830. The suture 800 is adapted to provide moment to adjacent spinous processes 830, pulling the spinal processes 830 together to cause gradual lordosis of the spine (FIG. 16). It should be noted that one or more sutures, including sutures 100, 200, 300, 400, 500, 700, or 800 may be used and that one or more fusion cage configurations, such as bullet cage 802, may be used to perform the PLIF procedure of FIGS. 12-16.

Referring to FIG. 17, an embodiment of a medical device kit includes one or more sutures 800, as shown in FIG. 11A, and a medical device 850 that is attached via the sutures 800 to an animal tissue 854. Elastomeric properties of the suture 800 can prevent suture breakage and detachment of the device during patient movement, and allow for articulation and other movement of the tissue to which the suture is attached, of tissues adjacent to the implantation site, and of the implanted device with respect to the tissue.

In one embodiment, the medical device 850 comprises a pump that delivers drugs to the intrathecal space for example. One or more sutures 800 are adapted to secure the pump to the underlying muscle fascia 854 by passing the suture through an anchor 852 in the device 850 and through an opening in the tissue 854. In some embodiments, the distal end 102 of the suture 800 has a piercing element 124, such as a needle. The distal end 102 can be used as a lasso and wrapped around, and can be passed through the anchor 852 in the medical device 850 and the animal tissue 854, and looped back through the aperture 216 to secure the medical device 850 to the animal tissue 854 within the holding loop 218.

Other devices can by implanted and attached to tissue using the sutures disclosed herein, including analgesic implant device, providing vibratory massage to a tissue 854 located adjacent to the medical device 850. The elastomeric properties of the sutures 800 maintain the device 850 in direct contact with the tissue 854 to provide the analgesic effect. While suture 800 is exemplified in FIG. 17, in other embodiments other sutures may be used, including 100, 200, 300, 400, 500, and 700.

It should be appreciated that any number of sutures can be deployed to secure together two or more tissue portions 150, 152 or medical devices 850. The sutures, such as sutures 100, 200, 300, 400, 500, 700, or 800 can be suitably spaced one from another, to facilitate suitable contact and/or connection between the tissue portions 150, 152 secured thereby. It should also be appreciated that some embodiments of sutures can include tissue capture portions as disclosed with respect to other sutures. For example, a suture can include a tissue capture portion as discussed with respect to suture 400 and another tissue capture portion as discussed with respect to suture 500, or the like.

While the foregoing is directed to certain embodiments, other and further embodiments can be devised without departing from the basic scope thereof, and the scope thereof is determined by the claims that follow. While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. A suture, comprising:
an elongated body made of an elastomeric material; and
a surgical needle connected to the distal end of the elongate body and configured for piercing animal tissue.

2. The suture of claim 1, wherein the elongated body comprises:
a filament extending along a longitudinal axis and having a filament radial dimension; and
a protrusion protruding radially from the filament and being made of an elastomeric material selected such that axial tension causes the protrusion to narrow.

3. A suture, comprising:
an elongate body, including:
a filament extending along a longitudinal axis and having a filament radial dimension sized to extend through a tissue opening through animal tissue, and
a protrusion connected to the filament and protruding radially with respect thereto, the protrusion defining a longitudinal end of a capture filament section of the filament, which protrusion has a radial dimension that is sufficiently larger than the filament radial dimension and is made of an elastomeric material selected such that axial tension causes the protrusion to narrow to a narrowed radial dimension that is sufficiently reduced to allow the protrusion to be pulled through the opening to position the capture filament section to move into the tissue opening; and
a piercing element connected to the elongate body and configured for piercing the tissue to make the opening.

4. The suture of claim 3, wherein:
the protrusion comprises a distal capture element; and
the elongate body includes a proximal capture element protruding radially at an opposite longitudinal end of the capture filament section, which proximal capture element has a capture element radial dimension that is sufficiently larger than the filament radial dimension to prevent the proximal capture element from sliding into the opening to thereby capture the tissue therebetween on the capture filament section for suturing the tissue.

5. The suture of claim 4, wherein the elongate body includes a filament that has:
the capture filament section; and
a distal filament section disposed distally of the capture section, which distal filament section has a radial dimension that is smaller than the capture element radial dimension and sized to allow the distal filament section to be pulled through the opening.

6. The suture of claim 5, wherein the distal capture element is a nodule that is proud of the distal filament section and the capture section.

7. The suture of claim 5, wherein the filament includes a proximal filament section disposed proximally of the capture filament section and has a radial dimension that is smaller than the capture element radial dimension, the proximal filament section being sufficiently long to allow handling to apply axial tension across the elongate body to cause the distal capture element to narrow to the narrowed radial dimension.

8. The suture of claim 7, wherein the proximal and distal capture elements are nodules that are proud of the proximal filament section and the capture section.

9. The suture of claim 5, wherein the distal capture element includes a plurality of bent expansive portions that straighten elastically for contracting and bend elastically for re-expanding radially, and which are connect the distal filament section to the capture filament section.

10. The suture of claim 9, wherein the distal capture element includes a core that elastically maintains the plurality of expansive portions in a bent position.

11. The suture of claim 4, wherein the proximal capture element is made of an elastomeric material selected such that axial tension causes the distal capture element to narrow to a narrowed radial dimension that is sufficiently reduced to allow the distal capture element to be pulled through the opening.

12. The suture of claim 4, wherein the elastomeric material is selected such that the narrowing of the distal capture element is elastic, such that the distal capture element returns to the capture element radial dimension when the tension is released.

13. The suture of claim 4, wherein the capture elements have surfaces facing the capture filament section, which surfaces are sufficiently steep to a sufficient radial dimension to prevent the capture elements to slide through the opening when the tension is released.

15. The suture of claim 4, wherein the elongate body includes a visual indicator that indicates when the distal capture element has passed through the opening.

16. The suture of claim 15, wherein the visual indicator is configured to indicate when the tissue is positioned between the capture elements.

17. The suture of claim 4, wherein the distal capture element has a higher Poisson ratio than another portion of the elongate body.

18. The suture of claim 4, further comprising a manipulation protrusion protruding radially from the manipulation feature at a location proximal from the proximal capture element to provide purchase for a user to pull to apply the axial tension across the elongate body.

19. The suture of claim 3, further comprising a holding structure located on the elongate body and defining a holding aperture, the holding aperture having an inside dimension that sized to receive an opposite end of the elongate body therethrough to form the capture section, wherein the protrusion has a radial dimension such that the protrusion is capable of being pulled through the holding aperture, but sufficiently large to be retained by the holding structure against sliding back through the holding aperture to secure the suture.

20. The suture of claim 3, wherein the filament is made of an elastomeric material.

21. A suture, comprising:
an elongate body including:
a filament section extending along a longitudinal axis, and
a protrusion protruding radially with respect to the filament section to a protrusion radial dimension; and
a holding structure located on the elongate body and defining a holding aperture, the holding aperture having an inside dimension that is smaller than the protrusion;
wherein at least one of the holding structure or the protrusion is made of elastomeric material such that the protrusion can be pulled through the holding aperture by a user, but the protrusion radial dimension is sufficiently larger than the inside dimension of the aperture so that the holding structure retains the protrusion against sliding back through the holding aperture, thereby defining a capture section along the filament between the holding structure and the protrusion.

22. The suture of claim 21, wherein the protrusion is made of elastomeric material such that axial tension on the elongate body causes the protrusion to contract from the protrusion radial dimension to allow the protrusion to be pulled through the holding aperture.

23. The suture of claim 21, wherein the protrusion comprises a plurality of protrusions spaced along the filament, such that a selected number of the protrusions can be pulled through the holding aperture to selected a length of the capture section.

24. The suture of claim 21, further comprising a surgical needle connected to the elongate body on an opposite end from the holding structure across the capture section.

25. A kit for use in performing posterior lumbar interbody fusion comprising:
the suture of claim 21, wherein the suture body is made of an elastomeric material and is configured to secure to and apply elastic tension to spinal processes of adjacent vertebrae; and
a cage configured to be implanted between and fuse together the adjacent vertebrae.
